(19) United States
(12) Patent Application Publication
(10) Pub. No.: US 2026/0217663 A1
(43) Pub. Date: Jul. 30, 2026

(54) METHODS AND COMPOSITIONS FOR TREATING HSD-1-MEDIATED DISORDERS

(71) Applicant: Sparrow Pharmaceuticals, Inc., Portland, OR (US)

(72) Inventor: Jerry B. EVARTS, JR., Las Vegas, NV (US)

(21) Appl. No.: 19/144,670

(22) PCT Filed: Jan. 5, 2024
(86) PCT No.: PCT/US2024/010474

§ 371 Date: Jun. 30, 2025

Related U.S. Application Data
(60) Provisional application No. 63/478,792, filed on Jan. 6, 2023.

(51) Int. Cl. C07D 249/08; C07D 271/10

(52) U.S. Cl. CPC C07D 249/08; C07D 271/10

(57) ABSTRACT

Provided are processes for preparing triazole derivatives and compositions for use as HSD-1 inhibitors.

This application claims the benefit of priority of U.S. provisional application No. 63/478,792, filed Jan. 6, 2023, the contents of which are incorporated by reference as if written herein in their entirety.

Glucocorticoids (GCs) are corticosteroids that bind to the glucocorticoid receptor (GR), which is present in many cell types in the human body. GCs are involved in cardiovascular, metabolic, immunologic, osteal, muscular, dermatological, ocular, psychiatric, cognitive, circadian, and homeostatic functions. In addition, GCs can also bind to the mineralocorticoid receptor (MR) and non-genomic receptors.

Important natural GCs include cortisol (known medically as hydrocortisone) and corticosterone. Synthetic GCs include prednisolone, methylprednisolone, dexamethasone, and many others, as well as derivatives of these. Additionally, inactive congeners (e.g., cortisone, prednisone) that don't activate GR are commonly referred to as GCs. Both cortisol and synthetic GCs are used as medications to treat autoimmune diseases and other conditions. However, an excess of either natural or synthetic GCs in humans can lead to myriad symptoms and illnesses, including hyperglycemia, insulin resistance, obesity, hyperlipidemia, hypertension, and Cushing's syndrome. Such excesses are most commonly caused either by tumors that secrete cortisol or hormones that increase cortisol secretion (e.g., ACTH or CRH), or through excess administration of hydrocortisone or synthetic GCs during medical treatment.

11β-hydroxysteroid dehydrogenases (HSDs) are enzymes that regulate the intracellular levels of glucocorticoids. The HSD enzymes consist of two isoforms: the nicotinamide-adenine dinucleotide phosphate-dependent type 1 (HSD-1), which converts inactive cortisone to active cortisol, and the nicotinamide-adenine dinucleotide dependent oxidative type 2 (HSD-2), which converts cortisol to cortisone. HSD-1 is a major source of intracellular cortisol and is thought to be a major source of intracellular synthetic GC in many cell types. Excess intracellular GC activates GR and MR, along with non-genomic receptors, resulting in the tissue-specific morbidity observed in subjects with GC excess. Inhibition of HSD-1 may therefore ameliorate those symptoms.

Novel potent HSD-1 inhibitors, including 4-(5-(2-(4-chloro-2,6-difluorophenoxy) propan-2-yl)-4-methyl-4 h-1,2,4-triazol-3-yl)-3-fluorobenzamide and related compounds, have been described in U.S. Pat. No. 8,377,923, the contents of which are hereby incorporated by reference in their entirety, as inhibitors of HSD-1 with promising potential.

There exists a need for new and improved methods for the production of 4-(5-(2-(4-chloro-2,6-difluorophenoxy) propan-2-yl)-4-methyl-4 h-1,2,4-triazol-3-yl)-3-fluorobenzamide and related compounds which are amenable to large-scale, high-purity synthesis for use as HSD-1 inhibitors for the treatment of HSD-1-mediated diseases.

Citation of any reference throughout this application is not to be construed as an admission that such reference is prior art to the present application.

### SUMMARY

Provided is a compound of structural formula II

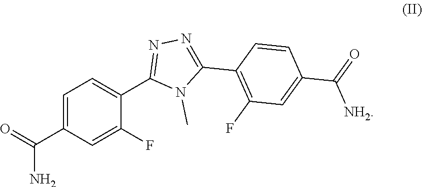 [SMILES: Cn1c(-c2ccc(C(N)=O)cc2F)nnc1-c1ccc(C(N)=O)cc1F]

Also provided is a compound of structural formula XIII:

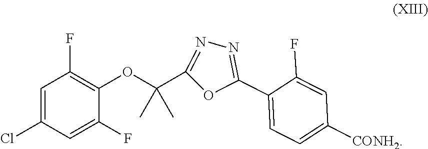 [SMILES: CC(C)(Oc1c(F)cc(Cl)cc1F)c1nnc(-c2ccc(C(N)=O)cc2F)o1]

Also provided is a compound of structural formula XIV:

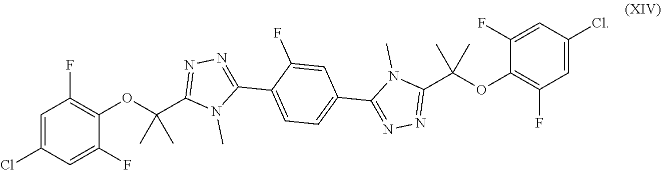 [SMILES: Cn1c(-c2ccc(-c3nnc(C(C)(C)Oc4c(F)cc(Cl)cc4F)n3C)c(F)c2)nnc1C(C)(C)Oc1c(F)cc(Cl)cc1F]

Also provided is a composition comprising a compound of structural formula I:

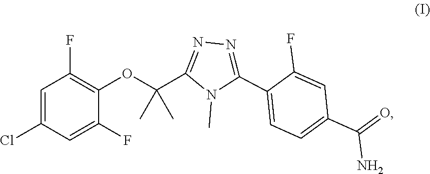 [SMILES: Cn1c(-c2ccc(C(N)=O)cc2F)nnc1C(C)(C)Oc1c(F)cc(Cl)cc1F]

or a salt thereof, wherein the composition contains a non-detectable amount of a compound of structural formula II:

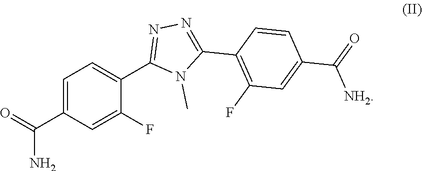 [SMILES: Cn1c(-c2ccc(C(N)=O)cc2F)nnc1-c1ccc(C(N)=O)cc1F]

Also provided is a composition comprising a compound of structural formula I:

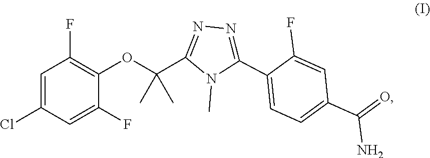 [SMILES: Cn1c(-c2ccc(C(N)=O)cc2F)nnc1C(C)(C)Oc1c(F)cc(Cl)cc1F]

or a salt thereof, wherein the composition contains a detectable amount of about 0.20% or less of a compound of structural formula XIII:

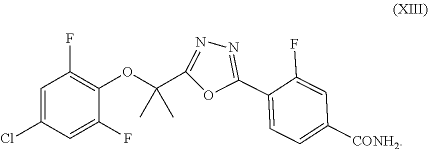 [SMILES: CC(C)(Oc1c(F)cc(Cl)cc1F)c1nnc(-c2ccc(C(N)=O)cc2F)o1]

Also provided is a composition comprising a compound of structural formula I:

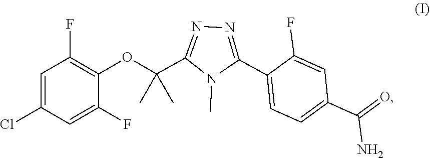 [SMILES: Cn1c(-c2ccc(C(N)=O)cc2F)nnc1C(C)(C)Oc1c(F)cc(Cl)cc1F]

or a salt thereof, wherein the composition contains a detectable amount of about 0.20% or less of a compound of structural formula XIV:

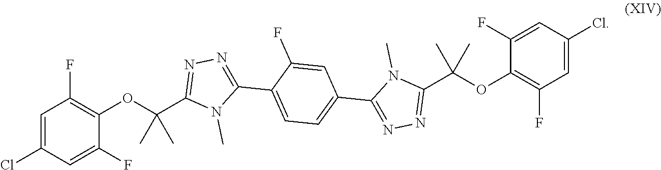 [SMILES: Cn1c(-c2ccc(-c3nnc(C(C)(C)Oc4c(F)cc(Cl)cc4F)n3C)c(F)c2)nnc1C(C)(C)Oc1c(F)cc(Cl)cc1F]

Also provided is a pharmaceutical composition comprising a composition of described herein, and a pharmaceutically acceptable carrier.

Also provided is a method for treating a HSD-1-mediated disorder in a subject in need thereof, comprising the step of administering to the subject a therapeutically effective amount of the composition or pharmaceutical composition described herein.

Also provided is a method for treating GC excess, or a condition thereof, in a subject in need thereof, comprising the step of administering to the subject a therapeutically effective amount of the composition or pharmaceutical composition described herein. Conditions of GC excess include Cushing's syndrome and autonomous cortisol secretion. GC excess may also be caused by the use of one or more GC medications.

These and other aspects of the disclosure disclosed herein will be set forth in greater detail as the patent disclosure proceeds.

### DETAILED DESCRIPTION

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant FIGURES (e.g., 1.255 μM, 2.1 μM, 2.9999 μM, etc.).

The term “about,” as used herein when referring to a measurable value such as an amount of a compound, dose, time, temperature, and the like, is meant to encompass variations of 20%, 10%, 5%, 1%, 0.5%, or even 0.1% from the specified amount.

The term “detectable” refers to a measurable quantity measured using an HPLC method having a detection limit of 0.05 area %.

The term “chlorinating reagent,” as used herein, refers to a compound or salt that adds a chlorine atom or atoms to an organic compound in a chemical reaction.

The term “hydrating agent,” as used herein, refers to a compound, salt, catalyst, or combination thereof that effects the net addition of one or more molecules of water to an organic compound in a chemical reaction.

The term “intermediate,” as used herein, refers to the major organic product of a chemical reaction, or a salt thereof, which is not isolated or purified (i.e., a “crude product”) before proceeding to the next step of the process.

The term “non-nucleophilic base,” as used herein, refers to a sterically hindered organic base that is a poor nucleophile. Examples of non-nucleophilic bases include N,N-diisopropylethylamine (DIPEA), 8-diazabicycloundec-7-ene (DBU), 1,5-diazabicyclo(4.3.0) non-5-ene (DBN), 2,6-dimethylpyridine (2,6-lutidine), 2,6-di-tert-butylpyridine, tert-butyl-lithium, tert-butyl-phosphazene, lithium diisopropylamide (LDA), sodium bis(trimethylsilyl)amide (NaHMDS), potassium tert-butoxide, potassium bis(trimethylsilyl)amide (KHMDS), lithium tetramethylpiperidide (LiTMP), sodium hydride, potassium hydride, sodium tert-butoxide, and potassium tert-butoxide.

The term “nucleophilic catalyst,” as used herein, refers to a Lewis base which catalyzes the reaction of a compound through the donation of an electron pair.

The term “polar solvent,” as used herein, refers to a solvent with large dipole moment.

The term “polar aprotic solvent,” as used herein, refers to a polar solvent that lacks an acidic hydrogen. Consequently, they are not hydrogen bond donors. Examples of polar aprotic solvents include acetone, acetonitrile, dichloromethane, dimethyl sulfoxide (DMSO) dimethylformamide (DMF), ethyl acetate, hexamethylphosphoric triamide (HMPT), pyridine, and tetrahydrofuran (THF).

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single dose unit having a fixed ratio of active ingredients or in multiple, separate dose units for each active ingredient. In some embodiments, the dose unit is a tablet. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder or on the effecting of a clinical endpoint.

The term “therapeutically acceptable” refers to those compounds (or salts) which are suitable for use in contact with the tissues of patients without undue toxicity, irritation, or allergic response, are commensurate with a reasonable benefit/risk ratio, and are effective for their intended use.

As used herein, “treating,” “treatment,” and the like means the administration of therapy to an individual who already manifests at least one symptom of a disease or condition or who has previously manifested at least one symptom of a disease or condition. For example, “treating” can include alleviating, abating, or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

The term “patient” is generally synonymous with the term “subject” and includes all animals including humans. Examples of patients include humans and primates, such as cynomolgus monkeys. Preferably, the patient is a human.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

In the Examples below and throughout the disclosure, the following abbreviations may be used: Boc=tert-butyloxycarbonyl; DMSO=dimethylsulfoxide; DCM=dichloromethane; DMAP=4-dimethylaminopyridine; DMF=dimethylformamide; EtOAc=ethyl acetate; EDC=1-ethyl-3-(3-dimethylaminopropyl) carbodiimide; EtOH=ethanol; TsOH=p-toluenesulfonic acid; ¹H-NMR=Proton Nuclear magnetic Resonance; HPLC=High Performance Liquid Chromatography; UPLC=Ultra Performance Liquid Chromatography; TLC=Thin Layer Chromatography. Other abbreviations may be used and will be familiar in context to those of skill in the art.

### Compounds and Compositions

Provided is a compound of structural formula II

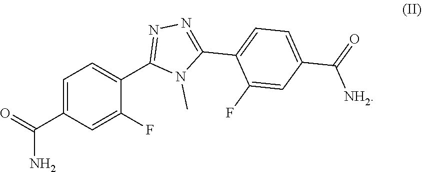 [SMILES: Cn1c(-c2ccc(C(N)=O)cc2F)nnc1-c1ccc(C(N)=O)cc1F]

Also provided is a composition comprising a compound of structural formula I:

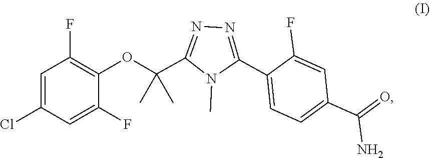 [SMILES: Cn1c(-c2ccc(C(N)=O)cc2F)nnc1C(C)(C)Oc1c(F)cc(Cl)cc1F]

or a salt thereof, wherein the composition contains a non-detectable amount of a compound of structural formula II:

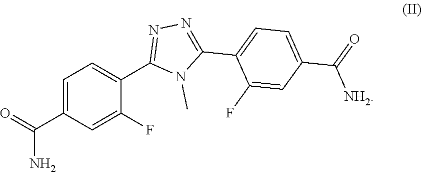 [SMILES: Cn1c(-c2ccc(C(N)=O)cc2F)nnc1-c1ccc(C(N)=O)cc1F]

Also provided is a compound of structural formula XIII:

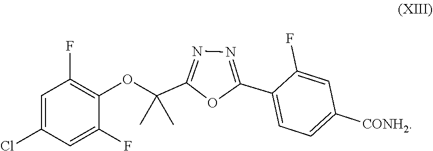 [SMILES: CC(C)(Oc1c(F)cc(Cl)cc1F)c1nnc(-c2ccc(C(N)=O)cc2F)o1]

Also provided is a composition comprising a compound of structural formula I:

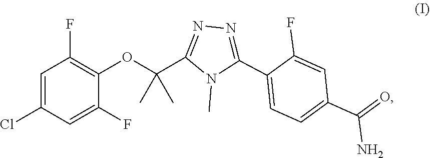 [SMILES: Cn1c(-c2ccc(C(N)=O)cc2F)nnc1C(C)(C)Oc1c(F)cc(Cl)cc1F]

or a salt thereof, wherein the composition contains a detectable amount of about 0.2% or less of a compound of structural formula XIII:

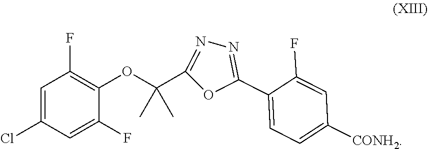 [SMILES: CC(C)(Oc1c(F)cc(Cl)cc1F)c1nnc(-c2ccc(C(N)=O)cc2F)o1]

Also provided is a compound of structural formula XIV:

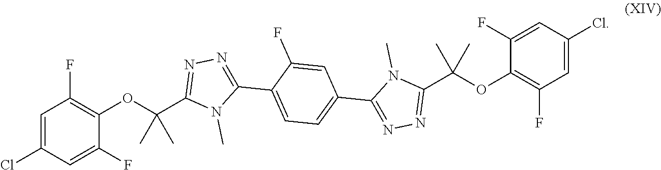 [SMILES: Cn1c(-c2ccc(-c3nnc(C(C)(C)Oc4c(F)cc(Cl)cc4F)n3C)c(F)c2)nnc1C(C)(C)Oc1c(F)cc(Cl)cc1F]

Also provided is a composition comprising a compound of structural formula I:

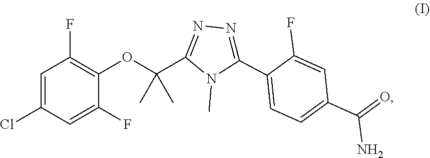 [SMILES: Cn1c(-c2ccc(C(N)=O)cc2F)nnc1C(C)(C)Oc1c(F)cc(Cl)cc1F]

or a salt thereof, wherein the composition contains a detectable amount of about 0.2% or less of a compound of structural formula XIV:

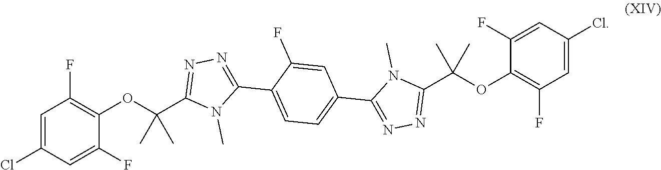 [SMILES: Cn1c(-c2ccc(-c3nnc(C(C)(C)Oc4c(F)cc(Cl)cc4F)n3C)c(F)c2)nnc1C(C)(C)Oc1c(F)cc(Cl)cc1F]

### Prior Art Process

U.S. Pat. No. 8,377,923 (′923) reports that the compound of Formula I, termed Example 186 in '923, was prepared using the process of Example 15 of '923 with the appropriate starting materials as shown in the following scheme.

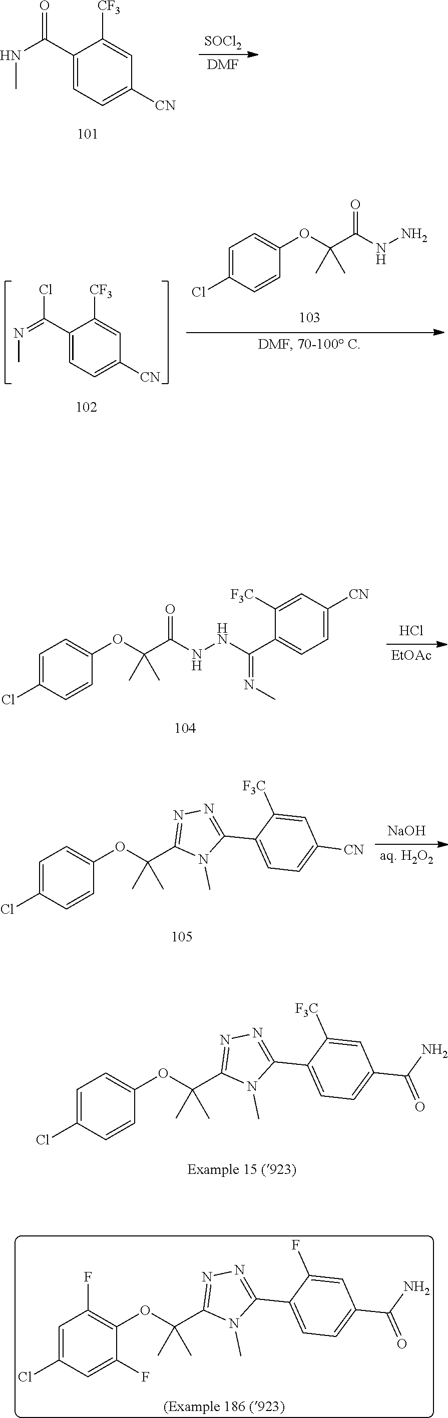

The coupling step was conducted using imidoyl chloride 102 and propanohydrazide 103 in a 1:1 ratio, utilizing DMF as the solvent without any additional base at a temperature of 70-100° C. The coupled product was subsequently cyclized to the triazole with HCl in ethyl acetate, then hydrated with sodium hydroxide and hydrogen peroxide. '923 does not indicate the purity or yield of either Example 15 or Example 186.

### Scale-Up Process

Subsequently, a process was developed for the large scale synthesis of a compound of Formula I. The coupling step was conducted with 1.2 equivalents of the imidoyl chloride in DMAc/H₂O with 1.5 eq. of lutidine as the base at a temperature of 0-10° C. While the increased amount of the imidoyl chloride improved yield, it was discovered that the process produced a detectable amount of a compound of structural formula II

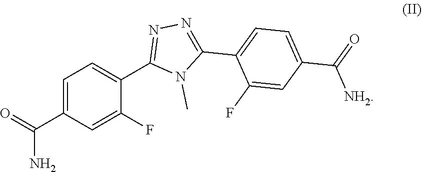 [SMILES: Cn1c(-c2ccc(C(N)=O)cc2F)nnc1-c1ccc(C(N)=O)cc1F]

that persisted through subsequent workup and purification steps to remain in the final product.

### Improved Scale-Up Process

A large-scale synthetic process has been discovered for the preparation of a composition of Formula I having a non-detectable amount of a compound of structural Formula II. The new process is based on the discovery that the formation of the compound of Formula II can be reduced if not completely suppressed by using dichloromethane as the solvent for the coupling step. The solvent switch enables higher reactivity between the starting materials, decreasing reaction time and allowing for a limited amount of the imidoyl chloride to be used while maintaining yields. Additionally, the favorable partition coefficient of dichloromethane allows additional impurities, including DMF-related impurities that form during the synthesis of the imidoyl chloride, to pass into the aqueous layer that is discarded during workup. The process comprises reacting a compound of structural Formula IV:

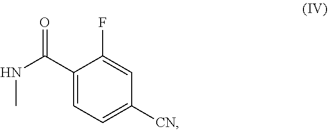

or a salt thereof, with a chlorinating reagent and a catalyst to form an intermediate of structural Formula V:

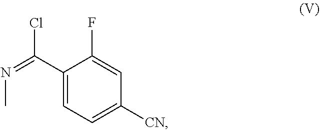

which is reacted with a compound of Formula III

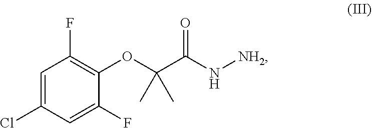 [SMILES: CC(C)(Oc1c(F)cc(Cl)cc1F)C(=O)NN]

and a non-nucleophilic base in dichloromethane to form a second intermediate of structural Formula VI:

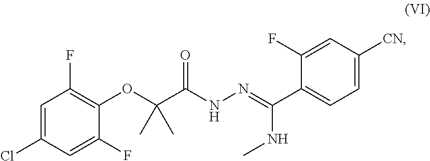 [SMILES: CN/C(=N\NC(=O)C(C)(C)Oc1c(F)cc(Cl)cc1F)c1ccc(C#N)cc1F]

which is reacted with a strong acid to form a triazole intermediate of structural Formula XII:

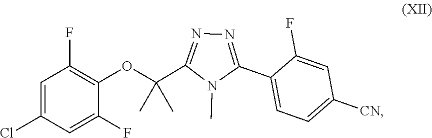 [SMILES: Cn1c(-c2ccc(C#N)cc2F)nnc1C(C)(C)Oc1c(F)cc(Cl)cc1F]

which is subjected to hydration conditions to yield the compound of Formula I.

In some embodiments, the chlorinating reagent is independently chosen from thionyl chloride, oxalyl chloride, and phosphoryl chloride.

In some embodiments, the chlorinating reagent is thionyl chloride.

In some embodiments, the catalyst is dimethylformamide.

In some embodiments, the chlorinating reagent is used in a molar excess of about 1.5:1 to 4:1 relative to the compound or intermediate of Formula IV or V.

In some embodiments, the non-nucleophilic base is chosen from N,N-diisopropylethylamine (DIPEA), triethylamine (TEA), 8-diazabicycloundec-7-ene (DBU), 1,5-diazabicyclo(4.3.0) non-5-ene (DBN), pyridine, 2,6-dimethylpyridine (2,6-lutidine), and 2,6-di-tert-butylpyridine.

In some embodiments, the non-nucleophilic base is 2,6-lutidine.

In some embodiments, the non-nucleophilic base is used in a molar excess of about 1.1:1 to 3:1 relative to the intermediate of Formula III.

In some embodiments, the strong acid is chosen from hydrochloric acid, nitric acid, sulfuric acid, hydrobromic acid, hydroiodic acid, perchloric acid, chloric acid, p-toluenesulfonic acid, trifluoroacetic acid, methanesulfonic acid, benzenesulfonic acid, and trifluoromethanesulfonic acid.

In some embodiments, the strong acid is p-toluenesulfonic acid.

In some embodiments, the hydration conditions are potassium carbonate and hydrogen peroxide.

In some embodiments, the compound of Formula III is prepared by reacting a compound of structural Formula VIII:

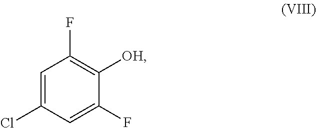

or a salt thereof, with ethyl 2-bromo-2-methylpropanoate and a base to form an intermediate of structural Formula IX:

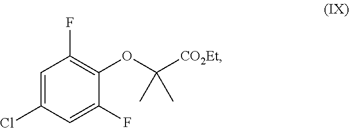 [SMILES: CCOC(=O)C(C)(C)Oc1c(F)cc(Cl)cc1F]

which is hydrolyzed to form an intermediate of structural Formula X:

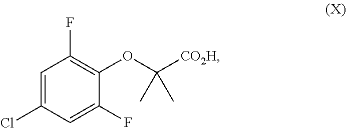 [SMILES: CC(C)(Oc1c(F)cc(Cl)cc1F)C(=O)O]

which is reacted with tert-butyl carbazate, a carbodiimide, and a nucleophilic catalyst to form a second intermediate of structural Formula XI:

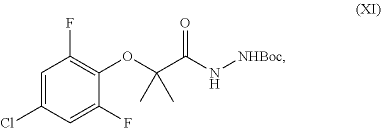 [SMILES: CC(C)(C)OC(=O)NNC(=O)C(C)(C)Oc1c(F)cc(Cl)cc1F]

that is subsequently reacted with a strong acid to form the compound of Formula III.

In some embodiments, the carbodiimide is chosen from N,N′-dicyclohexylcarbodiimide (DCC), N,N′-diisopropylcarbodiimide (DIC), and 1-ethyl-3-(3-dimethylaminopropyl) carbodiimide (EDC).

In some embodiments, the carbodiimide is EDC.

In some embodiments, the nucleophilic catalyst is chosen from 4-dimethylaminopyridine (DMAP) and hydroxybenzotriazole (HOBt).

In some embodiments, the nucleophilic catalyst is 4-dimethylaminopyridine (DMAP).

In some embodiments, the strong acid is chosen from hydrochloric acid, nitric acid, sulfuric acid, phosphoric acid, p-toluenesulfonic acid, trifluoroacetic acid, methanesulfonic acid, benzenesulfonic acid, and trifluoromethanesulfonic acid.

In some embodiments, the strong acid is hydrochloric acid.

In some embodiments, the ethyl 2-bromo-2-methylpropanoate is used in a molar excess of about 1.5:1 to 3:1 relative to the compound of Formula VIII.

In some embodiments, the base is chosen from sodium carbonate and potassium carbonate.

In some embodiments, the base is potassium carbonate.

In some embodiments, the base is used in a molar excess of about 1.5:1 to 3:1 relative to the compound of Formula VIII.

### Pharmaceutical Compositions

While it may be possible for the compounds and salts described herein to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, intranasal, pulmonary (including inhalation and nebulization), transdermal, rectal, and topical (including dermal, buccal, sublingual, and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound described herein or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

It should be understood that in addition to the ingredients particularly mentioned above, the formulations described above may include other agents conventional in the art having regard to the type of formulation in question, for example those suitable for oral administration may include flavoring agents.

The compounds and salts described herein may be administered orally or via injection at a dose of from 0.001 to 500 mg/kg per day. The dose range for adult humans is generally from 0.1 mg to 2 g/day. Tablets or other forms of presentation provided in discrete units may conveniently contain an amount of one or more compounds which is effective at such dosage or as a multiple of the same, for instance, units containing 0.05 mg to 500 mg, usually around 0.2 mg to 200 mg.

The amount of active ingredient that may be combined with the carrier materials to produce a single dosage form will vary depending upon the patient treated and the particular mode of administration.

The compounds and salts described herein can be administered in various modes, e.g., orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

### Indications and Methods of Treatment

Also provided are methods for treating HSD-1-mediated disorders in a human or animal subject in need of such treatment comprising administering to said subject an amount of a compound, or salt thereof, or composition thereof disclosed herein.

Also provided are methods for treating glucocorticoid excess, or a condition thereof, in a subject in need thereof, comprising the step of administering to the subject a therapeutically effective amount of a compound, or salt thereof, or composition thereof disclosed herein.

In some embodiments, the condition of glucocorticoid excess is Cushing's syndrome.

In some embodiments, the Cushing's syndrome may be caused by any of Cushing's disease, adrenal Cushing's syndrome, ectopic ACTH secretion, ectopic CRH secretion, or rare disorders such as Carney complex.

In some embodiments, the condition of glucocorticoid excess is autonomous cortisol secretion (also known as mild autonomous cortisol secretion, mild [autonomous] cortisol excess, subclinical Cushing's syndrome, or hidden hypercortisolism).

In some embodiments, the condition of glucocorticoid excess is caused by the use of one or more glucocorticoid medications.

Also provided is a method of reducing the severity of one or more side effects of treatment with one or more glucocorticoid medications in a subject.

In some embodiments, the side effect is selected from osteoporosis, avascular necrosis of bone, myopathy, hyperglycemia, diabetes mellitus, dyslipidemia, weight gain, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoporosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, and reactivation of latent infections.

In some embodiments, the HSD-1-mediated disorder is chosen from diabetes, non-alcoholic fatty liver disease, idiopathic intracranial hypertension, diabetic wound healing, hyperglycemia, insulin resistance, obesity, hyperlipidemia, and hypertension.

In some embodiments, the HSD-1-mediated disorder is chosen from diabetes, hyperlipidemia, non-alcoholic fatty liver disease, obesity, idiopathic intracranial hypertension, and diabetic wound healing.

Besides being useful for human treatment, certain compounds, salts, and formulations disclosed herein may also be useful for veterinary treatment of companion animals, exotic animals, and farm animals, including mammals, reptiles, and the like.

### EXAMPLES

### Synthetic Methods

The following invention is further illustrated by the following Examples. All IUPAC names were generated using CambridgeSoft's ChemDraw 21.0.

In the Examples below and throughout the disclosure, the following abbreviations may be used: Boc=tert-butyloxycarbonyl; DMSO=dimethylsulfoxide; DCM=dichloromethane; DMAP=4-dimethylaminopyridine; DMF=dimethylformamide; EtOAc=ethyl acetate; EDC=1-ethyl-3-(3-dimethylaminopropyl) carbodiimide; EtOH=ethanol; TsOH=p-toluenesulfonic acid; 1H-NMR=Proton Nuclear magnetic Resonance; HPLC=High Performance Liquid Chromatography; UPLC=Ultra Performance Liquid Chromatography; TLC=Thin Layer Chromatography. Other abbreviations may be used and will be familiar in context to those of skill in the art.

### Example 1

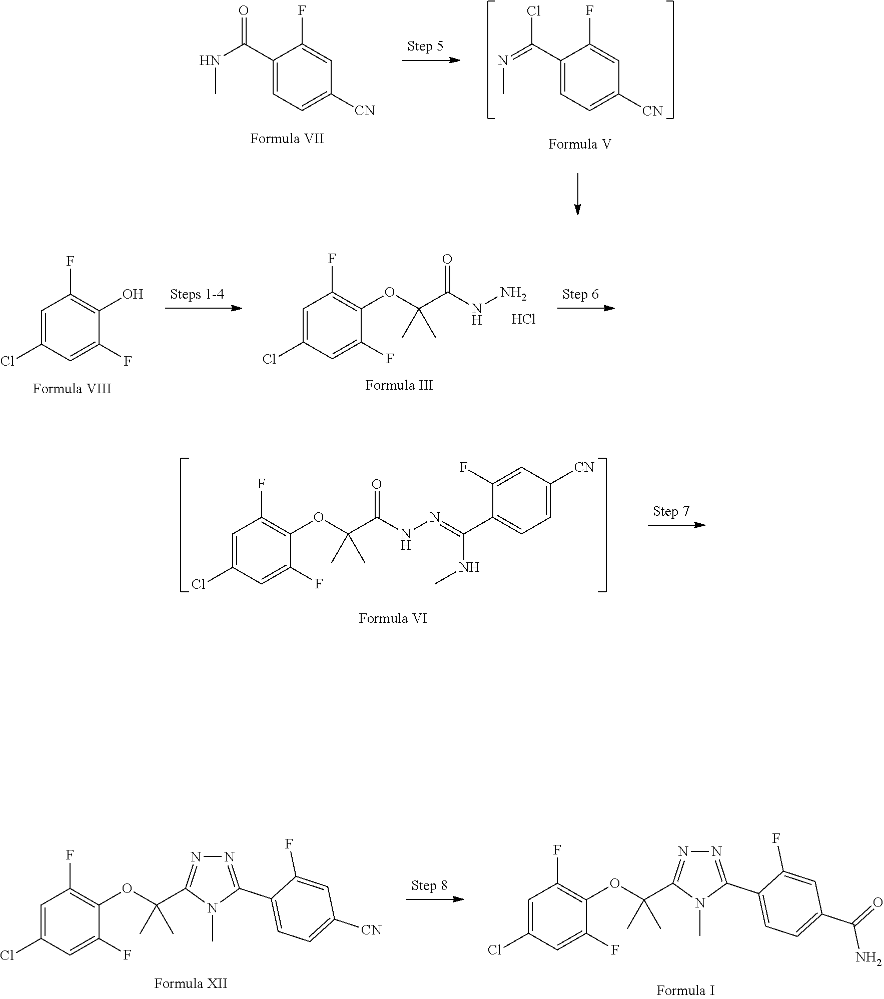

### Step 1

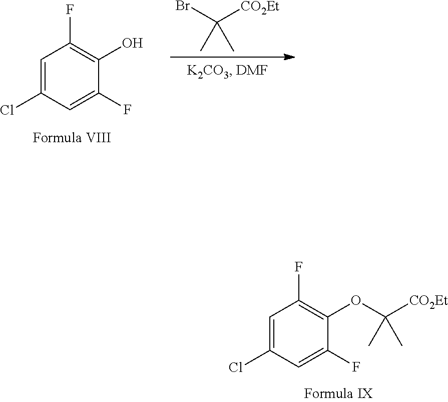

A mixture of 4-chloro-2,6-difluorophenol (Formula VIII, 1.0 eq.) and ethyl α-bromoisobutyrate (2.0 eq.) in DMF (5 volumes) was cooled to between 0-10° C. K₂CO₃ (1.5 eq.) in DMF (5 volumes) was added, and the mixture was raised to 50±5° C. and stirred for 20 hours. The reaction mixture was charged to a separate vessel containing water (10 volumes) and ethyl acetate (20 volumes). The organic layer was separated, neutralized with 1M HCl (10 volumes) and washed with 30% brine (10 volumes). The organic layer was then concentrated until about 2 volumes and solvent-switched with ethanol and concentrated until about 2 volumes. The crude ethanolic solution containing the compound of Formula IX was used without further purification in the next step.

### Step 2

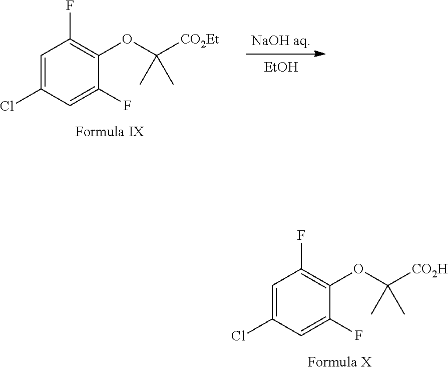 [SMILES: "EtOH" ; CCOC(=O)C(C)(C)Oc1c(F)cc(Cl)cc1F ; "NaOH aq." ; CC(C)(Oc1c(F)cc(Cl)cc1F)C(=O)O]

The crude ethanolic solution of Step 1 (2 volumes) was charged with an additional 10 volumes of EtOH and was cooled to between 0-10° C. NaOH (2.0 eq.) in water (7 volumes) was added, and the mixture was stirred for 1 hour at 25±5° C. The reaction mixture was neutralized with 6M HCl (2 volumes), then concentrated until about 7 volumes. Ethyl acetate (17 volumes) was added and stirred for 30 minutes, then the organic layer was separated, washed with 30% brine (10 volumes), concentrated, solvent-switched with ethyl acetate followed by acetonitrile, then concentrated again to approximately 3.5 volumes. The crude solution containing the compound of Formula X was used without further purification in the next step.

### Step 3

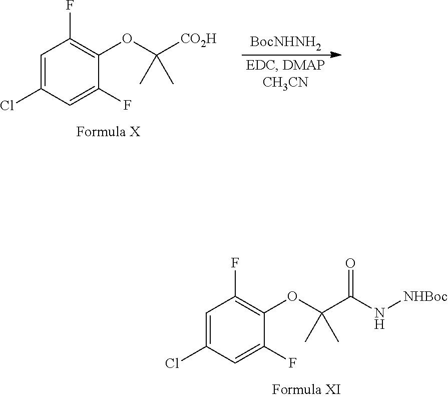 [SMILES: "BocNHNH2" ; CC(C)(Oc1c(F)cc(Cl)cc1F)C(=O)O ; "EDC, DMAPCH3CN" ; CC(C)(C)OC(=O)NNC(=O)C(C)(C)Oc1c(F)cc(Cl)cc1F]

The crude acetonitrile solution of Step 2 (3.5 volumes) was charged with t-butyl carbazate (1.05 eq), DMAP (0.05 eq), and an additional 7 volumes of acetonitrile. The mixture was cooled to between 0-10° C. and charged with EDC (1.2 eq), then stirred for 1 hour at 25±5° C. The reaction mixture was concentrated until about 3 volumes. Ethyl acetate (23 volumes) and 0.5M HCl (7.5 volumes) was added and stirred for 15 minutes. The organic layer was separated, washed with 30% brine (10 volumes), concentrated, solvent-switched with ethyl acetate, then concentrated again to approximately 3 volumes. The crude solution containing the compound of Formula XI was used without further purification in the next step.

### Step 4

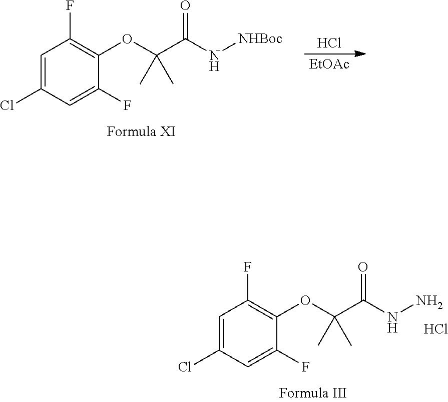 [SMILES: "EtOAc" ; CC(C)(C)OC(=O)NNC(=O)C(C)(C)Oc1c(F)cc(Cl)cc1F ; Cl ; CC(C)(Oc1c(F)cc(Cl)cc1F)C(=O)NN ; Cl]

The crude ethyl acetate solution of Step 3 (3 volumes) was dissolved in an additional 2 volumes of ethyl acetate, then charged to a 4N solution of HCl in ethyl acetate (4.5 eq) at 0-10° C. and stirred for 20 hours at 25±5° C. The reaction mixture was then concentrated until about 2 volumes. Ethyl acetate (2 volumes) was added and the reaction mixture was stirred for 30 minutes. The resulting crystals were filtered, washed with ethyl acetate (4 volumes), and dried in a vacuum oven at 40° C. to afford the compound of Formula III (90.9% yield overall for steps 1-4).

### Step 5

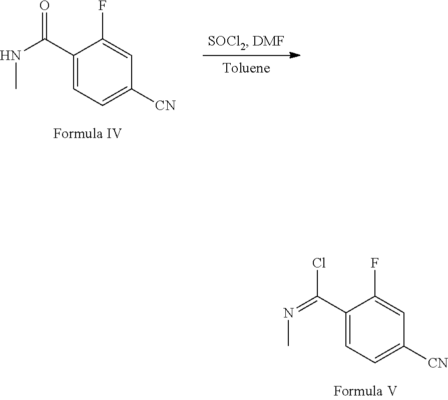

A compound of 4-cyano-2-fluoro-N-methylbenzamide (Formula IV, 1.0 eq) was charged to a reaction vessel, along with SOCl₂ (2.0 eq), DMF (0.1 eq), and toluene (6 volumes). The mixture was heated to between 75±5° C. and stirred for 2 hours, then heated to 100±5° C. and stirred for an additional 16 hours. The reaction mixture was concentrated until about 2.5 volumes. The mixture was redissolved in 6 volumes of toluene, concentrated until about 1.5 volumes, then dissolved in DCM (3.5 volumes). The crude solution containing the compound of Formula V was used without further purification in the next step.

### Step 6

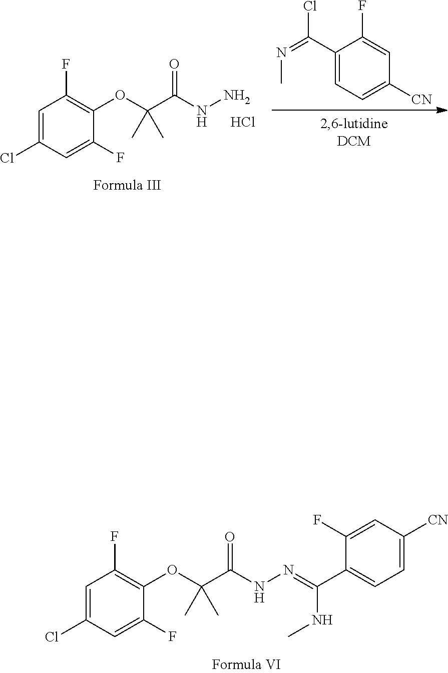 [SMILES: CC(C)(Oc1c(F)cc(Cl)cc1F)C(=O)NN ; Cl ; C/N=C(/Cl)c1ccc(C#N)cc1F ; CN/C(=N\NC(=O)C(C)(C)Oc1c(F)cc(Cl)cc1F)c1ccc(C#N)cc1F]

A reaction vessel was charged with a compound of Formula III (Step 4, 1.0 eq), 2.6-lutidine (1.5 eq), and acetonitrile (5 volumes). The mixture was cooled to −10±5° C. and the crude DCM solution of Step 6 (1.05 eq) was added dropwise. 5% aqueous NaHCO₃ (6 volumes) was added dropwise for 4 hours, then stirred for 1 hour at −5±5° C. The organic layer was separated, and the aqueous layer was washed with DCM (3.5 volumes). The combined organic layers were washed with 20% brine (6 volumes) and separated again. The crude solution containing the compound of Formula VI was used without further purification in the next step.

### Step 7

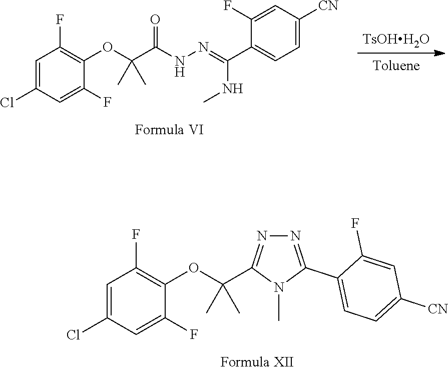

The crude DCM solution of Step 7 was charged with aqueous TsOH (0.05 eq), concentrated until about 1.5 volumes under vacuum at 20±5° C., then charged with toluene (7 volumes). The reaction mixture was heated to 90±10° C. and stirred for 1.5 hours, then cooled to 50±10° C. 5% aqueous NaHCO₃ (6 volumes) was added dropwise for 6 hours, then stirred for 40 minutes. Water (6 volumes) was added dropwise for 4 hours, then stirred for an additional 40 minutes. The organic phase was separated, concentrated, and recrystallized from ethanol and water. The resulting crystals were filtered, washed with a mixture of ethanol and water, and dried in a vacuum oven at 50° C. to afford the compound of Formula XII as a brown solid (46.3% yield overall for steps 7-8, 95.96% purity).

### Step 8

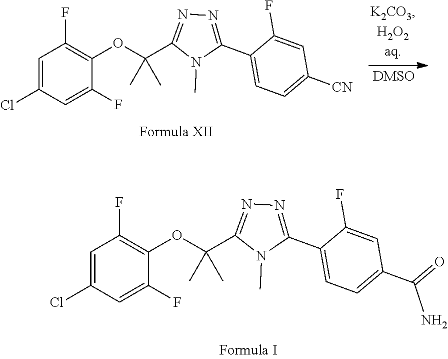 [SMILES: Cn1c(-c2ccc(C#N)cc2F)nnc1C(C)(C)Oc1c(F)cc(Cl)cc1F ; Cn1c(-c2ccc(C(N)=O)cc2F)nnc1C(C)(C)Oc1c(F)cc(Cl)cc1F]

A mixture of Formula XII (Step 8) in DMSO (8 volumes) was heated to 50±5° C. and stirred for 1 hour. The mixture was cooled to 25±5° C. and charged with K₂CO₃ (0.5 eq). 30% aqueous H₂O₂ (1.5 eq.) was added dropwise for 2 hours, then stirred for an additional 30 minutes. The reaction mixture was charged with 15.7% aqueous Na₂SO₃ (1.2 volumes), stirred for 1 hour, then charged with softened water (6.5 volumes) and stirred for an additional 2-3 hours. The solids were filtered, washed with water, dried in a vacuum oven at 45±5° C., and recrystallized from a mixture of ethanol and water to afford the compound of Formula I as an off-white solid (87.8% yield, 99.73% purity).

### Example 2

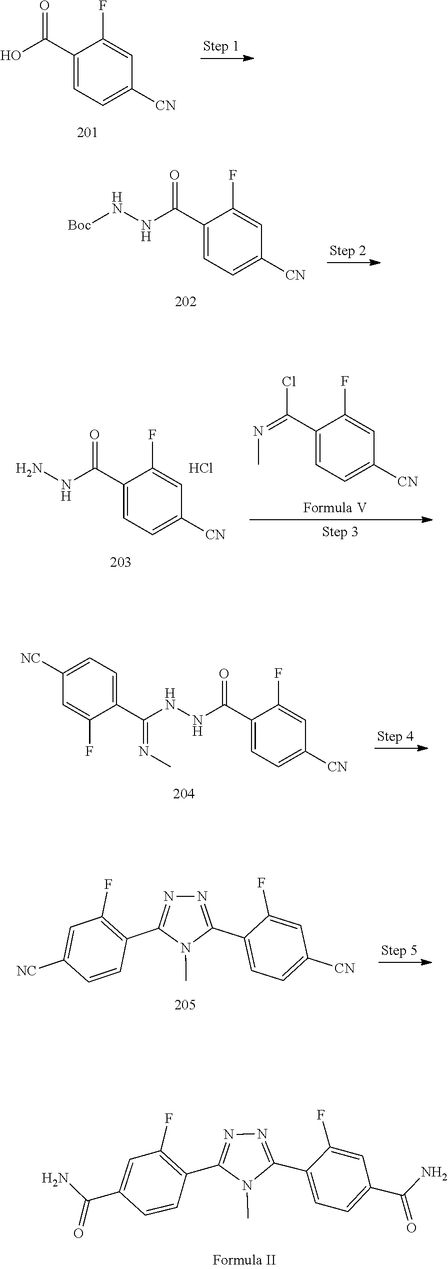

### Step 1

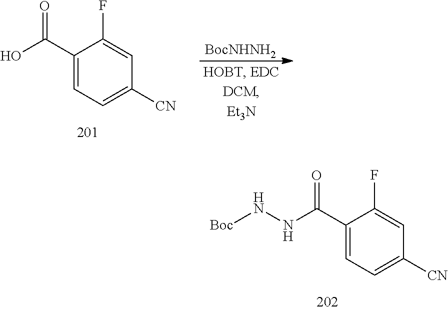 [SMILES: "BocNHNH2" ; N#Cc1ccc(C(=O)O)c(F)c1 ; CC(C)(C)OC(=O)NNC(=O)c1ccc(C#N)cc1F]

A mixture of compound 201 (1.0 eq), tert-butyl carbazate (1.2 eq), HOBT (1.2 eq), EDC (1.2 eq), and triethylamine (1.5 eq) in DCM (5 volumes), was stirred at 25±5° C. until completion. The reaction mixture was charged with water (8 volumes) and stirred for 10 minutes. The organic layer was separated, washed with 35% citric acid until the pH of the solution was between 6 and 7, washed with 25% NaHCO₃ until the pH of the solution was between 7 and 8, washed with water, and concentrated. The crude solid containing the compound 202 was used without further purification in the next step.

### Step 2

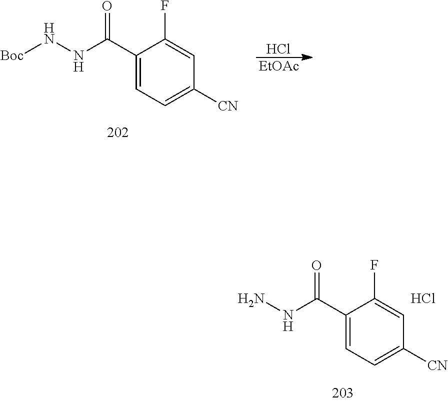 [SMILES: "EtOAc" ; CC(C)(C)OC(=O)NNC(=O)c1ccc(C#N)cc1F ; Cl ; N#Cc1ccc(C(=O)NN)c(F)c1 ; Cl]

A mixture of crude compound 202 (Example 2, Step 1, 1.0 eq), ethyl acetate (18 volumes), and a 4M solution of HCl in ethyl acetate (34 eq) was stirred at 25±5° C. until completion. The reaction mixture was concentrated, and the crude solid containing the salt 203 was used without further purification in the next step.

### Step 3

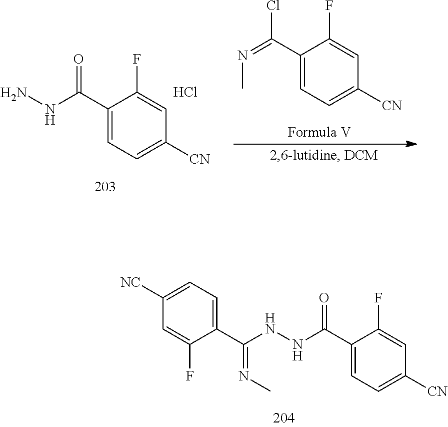 [SMILES: N#Cc1ccc(C(=O)NN)c(F)c1 ; Cl ; C/N=C(/Cl)c1ccc(C#N)cc1F ; C/N=C(\NNC(=O)c1ccc(C#N)cc1F)c1ccc(C#N)cc1F]

A mixture of crude salt 203 (Example 2, Step 2, 1.0 eq.), 2,6-lutidine (2.5 eq), and dichloromethane (5 volumes) was charged to a flask and cooled to −10±5° C. A solution of Formula V in dichloromethane (1.1 eq, 2.3 volumes) was added to the reaction mixture. The mixture was stirred at −10±5° C. for 16 h. 3% aqueous NaHCO₃ (40 volumes) was added to the reaction, then stirred for 1 hour at 10±5° C. The mixture was filtered, the cake was washed with water, and toluene (5 volumes) was charged to the cake. The resulting solution of compound 204 was used without further purification in the next step.

### Step 4

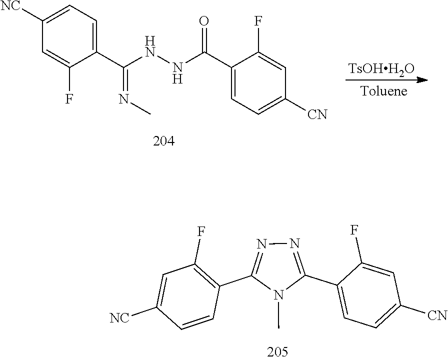 [SMILES: C/N=C(\NNC(=O)c1ccc(C#N)cc1F)c1ccc(C#N)cc1F ; "TsOH·H2O" ; Cn1c(-c2ccc(C#N)cc2F)nnc1-c1ccc(C#N)cc1F]

A solution of compound 204 (Example 2, Step 3, 1.0 eq) was charged with p-toluenesulfonic acid monohydrate (0.05 eq), warmed to 95±5° C., and stirred for 60 h. The reaction mixture was filtered, the cake washed with toluene, and the resulting crude solid 205 was used without further purification in the next step.

### Step 5

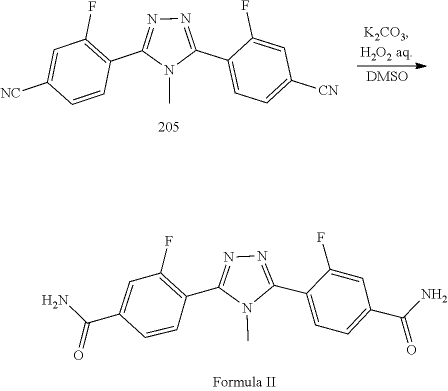 [SMILES: Cn1c(-c2ccc(C#N)cc2F)nnc1-c1ccc(C#N)cc1F ; Cn1c(-c2ccc(C(N)=O)cc2F)nnc1-c1ccc(C(N)=O)cc1F]

A mixture of crude compound 205 (Example 2, Step 4, 1.0 eq) DMSO (10 volumes) and charged with K₂CO₃ (0.5 eq) was charged to a round bottom flask. 30% aqueous H₂O₂ (3 eq) was added dropwise, then stirred at 15±5° C. for 16 h. The reaction mixture was charged with 15.7% aqueous Na₂SO₃ (20 volumes) dropwise. The solids were filtered, washed with acetonitrile, dried in a vacuum oven at 45±5° C., re-precipitated from a mixture of DMF and water, filtered, and dried again to afford the compound of Formula II as an off-white solid (91.51% purity).

### Example 3

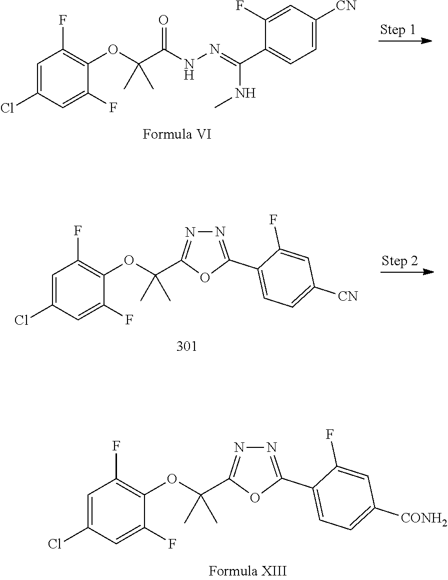 [SMILES: CN/C(=N\NC(=O)C(C)(C)Oc1c(F)cc(Cl)cc1F)c1ccc(C#N)cc1F ; CC(C)(Oc1c(F)cc(Cl)cc1F)c1nnc(-c2ccc(C#N)cc2F)o1 ; CC(C)(Oc1c(F)cc(Cl)cc1F)c1nnc(-c2ccc(C(N)=O)cc2F)o1]

### Step 1

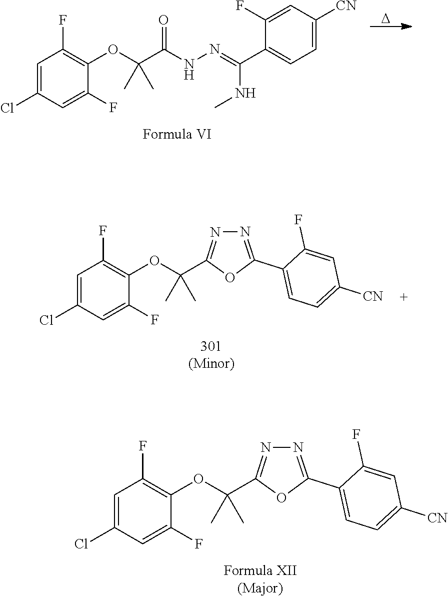

A solution containing a compound of Formula VI (Example 1, Step 6, 1.0 eq) in dimethylacetamide was heated to 90±10° C. and stirred for 3 h. The mixture was purified using column chromatography to afford compound 301 in 10.04% yield and a compound of Formula XII in 55.7% yield.

### Step 2

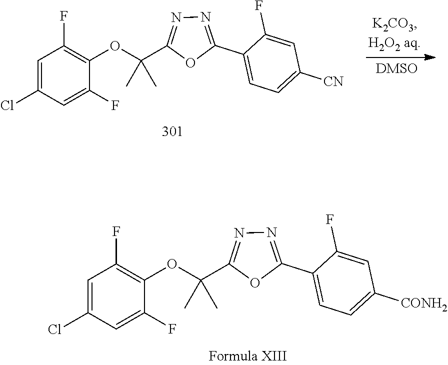 [SMILES: CC(C)(Oc1c(F)cc(Cl)cc1F)c1nnc(-c2ccc(C#N)cc2F)o1 ; CC(C)(Oc1c(F)cc(Cl)cc1F)c1nnc(-c2ccc(C(N)=O)cc2F)o1]

A mixture of compound 301, DMSO (20 volumes) and charged with K₂CO₃ (0.5 eq) was charged to a round bottom flask. 30% aqueous H₂O₂ (3 eq) was added dropwise, then stirred at 25±5° C. for 20 h. The reaction mixture was charged with water and stirred. The solids were filtered, washed with water, and dried in a vacuum oven at 45±5° C. to afford the compound of Formula XIII as an off-white solid (94.9% purity).

### Example 4

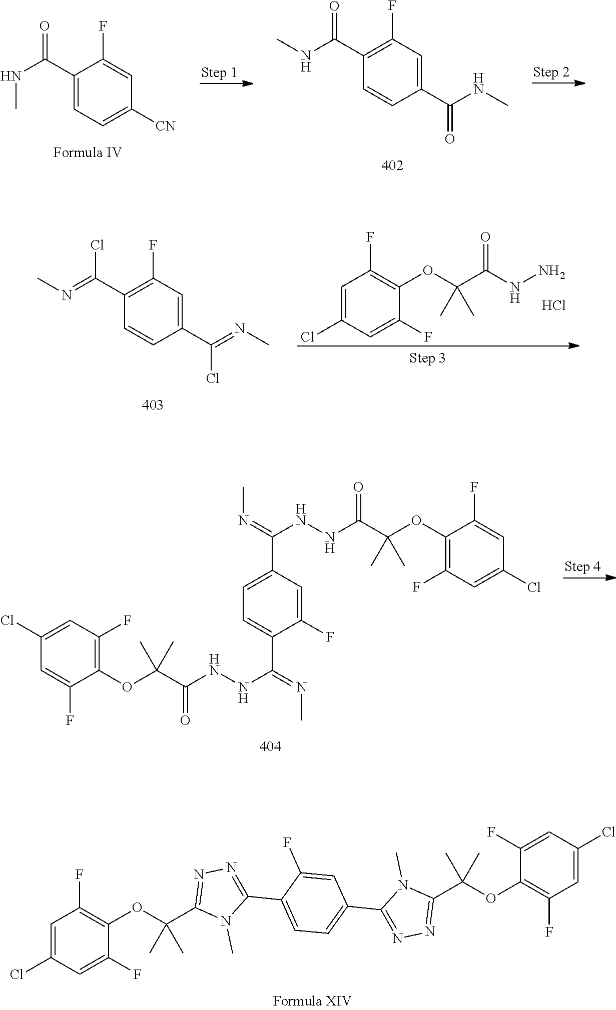

### Step 1

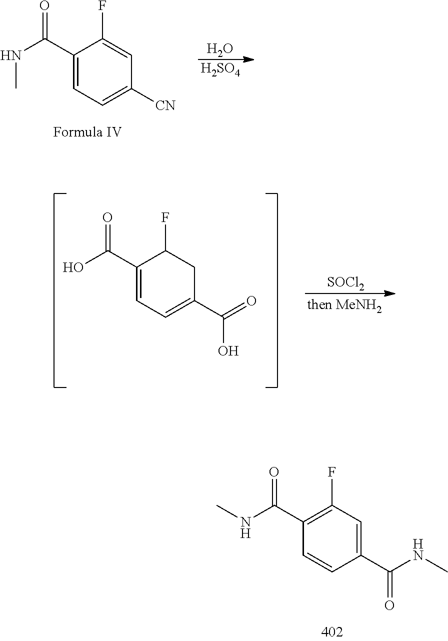

A mixture of a compound of Formula IV (1.0 eq) in water (3 volumes) was charged to a round bottom flask. Sulfuric acid (3 volumes) was added dropwise, and the mixture was heated to 90±10° C. and stirred for 64 h. Water (5 volumes) was added, and the solids were filtered and washed with water until the filtrate pH was between 5 and 7. The solids were dried at 50° C. for 24 h.

The solid intermediate was charged to a reactor with toluene (10.5 volumes) and thionyl chloride (3 eq). The mixture was heated to 75±5° C., stirred until clear, then stirred for an additional 16 h at 100° C. The mixture was concentrated and charged with acetonitrile (10 volumes), then added to a mixture of acetonitrile (7 volumes) and 40% methylamine in water (9 volumes) and stirred at 25° C. for 3 h. The mixture was adjusted to between pH 6 and 7 with 6M HCl, washed with ethyl acetate, concentrated, and recrystallized from ethanol to afford compound 402, which was used without further purification in the next step.

### Step 2

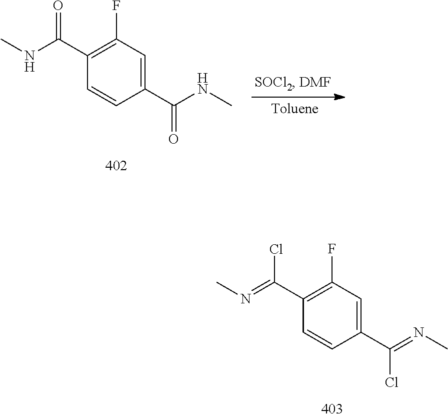

A solution containing compound 402 (1.0 eq), DMF (0.1 eq), thionyl chloride (3 eq) and toluene (6 volumes) was heated to 75±5° C. and stirred for 3 h, then heated to 100±5° C. and stirred for 16 h. The mixture was cooled to 55° C., concentrated, and charged with DCM (5 volumes) to afford a solution of compound 403, which was used in the next step without further purification.

### Step 3

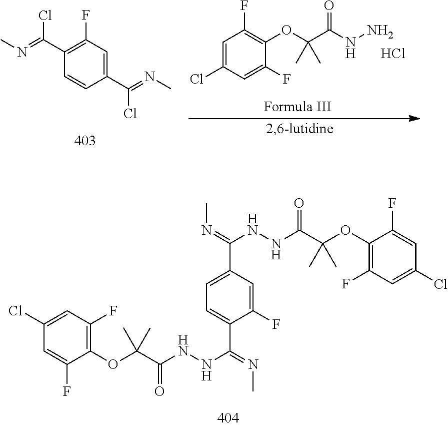

A reaction vessel was charged with a compound of Formula III (Example 1, Step 4, 1.0 eq), 2.6-lutidine (1.5 eq), and acetonitrile (5 volumes). The mixture was cooled to 5±5° C. and the crude DCM solution of 403 (1.05 eq) was added dropwise and stirred for 16 h. 5% aqueous NaHCO₃ (6 volumes) was added dropwise. The organic layer was separated, and the aqueous layer was washed with DCM (3.5 volumes). The combined organic layers were washed with water (6 volumes) and separated again. The crude solution containing compound 404 was used without further purification in the next step.

### Step 4

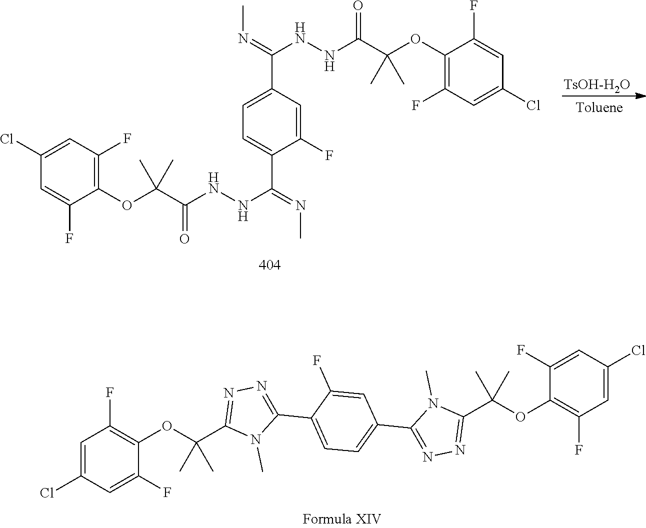

A solution of compound 404 (Step 3, 1.0 eq) was charged with p-toluenesulfonic acid monohydrate (0.05 eq), concentrated, charged with toluene (3 volumes), warmed to 90±10° C., and stirred for 16 h. The reaction mixture was filtered, and the solids were charged with DCM and stirred until clear. The solution was washed with 5% NaHCO₃, washed with water, concentrated, redissolved in hot ethanol, charged with water, filtered, and dried under vacuum to afford the compound of Formula XIV as a white solid (98.88% purity).

### HPLC Test Method Parameters for Identity and Impurity Analyses

The analytical test procedure used to test the identity and impurities of compounds employs reversed phase HPLC with gradient elution. The method is validated and has been shown to be stability indicating. Chromatographic separation was performed using a Waters Xterra RP18 column (150×4.6 mm, 5 μm, Waters Corporation, Milford, U.S.A.) and a Ghost-Sniper 4.6×50 mm ghost trapping column at 40° C.

The solvent system mobile phases were (A) sodium phosphate buffer, pH 7.0 and (B) acetonitrile. Samples were dissolved in a 50:50 mixture of A and B, 20 μL injection volume, then separated using the following gradient profile at 1.0 mL/min flow rate: (95% A, 5% B) linear change to (30% A, 70% B) over 50 minutes; (30% A, 70% B) for 10 minutes; and (95% A, 5% B) for 5 minutes. Compounds were detected at a wavelength of 220 nm.

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

All references, patents or applications, U.S. or foreign, cited in the application are hereby incorporated by reference as if written herein in their entireties. Where any inconsistencies arise, material literally disclosed herein controls.

What is claimed is:

1. A compound of structural formula II:

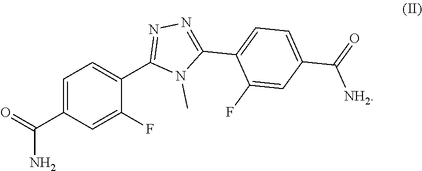 [SMILES: Cn1c(-c2ccc(C(N)=O)cc2F)nnc1-c1ccc(C(N)=O)cc1F]

2. A compound of structural formula XIII:

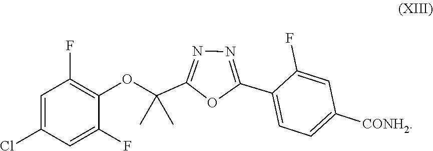 [SMILES: CC(C)(Oc1c(F)cc(Cl)cc1F)c1nnc(-c2ccc(C(N)=O)cc2F)o1]

3. A compound of structural formula XIV:

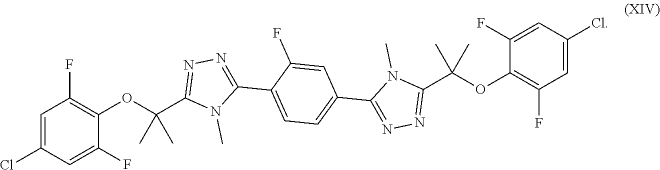 [SMILES: Cn1c(-c2ccc(-c3nnc(C(C)(C)Oc4c(F)cc(Cl)cc4F)n3C)c(F)c2)nnc1C(C)(C)Oc1c(F)cc(Cl)cc1F]

4. A composition comprising a compound of structural formula I: 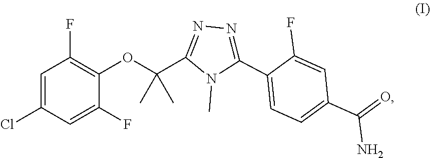 [SMILES: Cn1c(-c2ccc(C(N)=O)cc2F)nnc1C(C)(C)Oc1c(F)cc(Cl)cc1F]
or a salt thereof, wherein the composition contains a non-detectable amount of a compound of structural formula II:

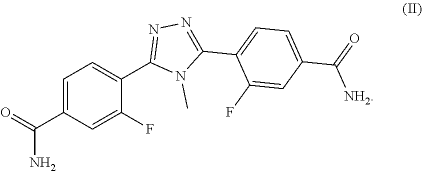 [SMILES: Cn1c(-c2ccc(C(N)=O)cc2F)nnc1-c1ccc(C(N)=O)cc1F]

5. A composition comprising a compound of structural formula I: 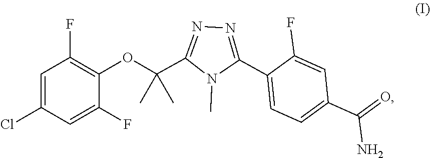 [SMILES: Cn1c(-c2ccc(C(N)=O)cc2F)nnc1C(C)(C)Oc1c(F)cc(Cl)cc1F]
or a salt thereof, wherein the composition contains a detectable amount of about 0.20% or less of a compound of structural formula XIII:

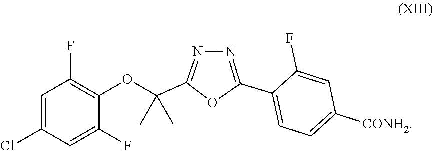 [SMILES: CC(C)(Oc1c(F)cc(Cl)cc1F)c1nnc(-c2ccc(C(N)=O)cc2F)o1]

6. A composition comprising a compound of structural formula I: 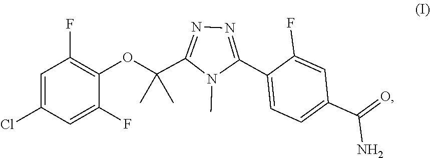 [SMILES: Cn1c(-c2ccc(C(N)=O)cc2F)nnc1C(C)(C)Oc1c(F)cc(Cl)cc1F]
or a salt thereof, wherein the composition contains a detectable amount of about 0.20% or less of a compound of structural formula XIV:

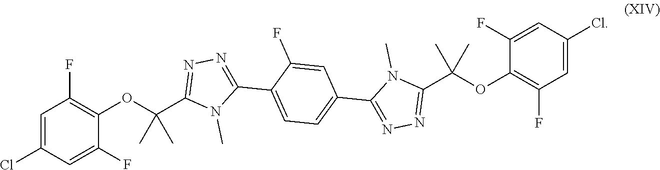 [SMILES: Cn1c(-c2ccc(-c3nnc(C(C)(C)Oc4c(F)cc(Cl)cc4F)n3C)c(F)c2)nnc1C(C)(C)Oc1c(F)cc(Cl)cc1F]

7. A pharmaceutical composition comprising a composition of any one of claims 4 to 6, and a pharmaceutically acceptable carrier.

8. A method for treating a HSD-1-mediated disorder in a subject in need thereof, comprising the step of administering to the subject a therapeutically effective amount of the composition of any one of claims 4 to 6, or the pharmaceutical composition of claim 7.

9. A method for treating glucocorticoid excess, or a condition thereof, in a subject in need thereof, comprising the step of administering to the subject a therapeutically effective amount of the composition of any one of claims 4 to 6, or the pharmaceutical composition of claim 7.

10. The method of claim 9, wherein the condition of glucocorticoid excess is Cushing's syndrome.

11. The method of claim 9, wherein the condition of glucocorticoid excess is autonomous cortisol secretion.

12. The method of claim 9, wherein the condition of glucocorticoid excess is caused by the use of one or more glucocorticoid medications.
